SEQUENCE LISTING (51 sequences)

SEQ ID NO: 1 - protein, 4 aa, synthetic construct
GQPR

SEQ ID NO: 2 - protein, 4 aa, synthetic construct
AQTR

SEQ ID NO: 3 - protein, 4 aa, synthetic construct
RSRK

SEQ ID NO: 4 - protein, 4 aa, synthetic construct
QTAV

SEQ ID NO: 5 - protein, 4 aa, synthetic construct
EKGD

SEQ ID NO: 6 - protein, 4 aa, synthetic construct
ELGD

SEQ ID NO: 7 - protein, 4 aa, synthetic construct
EAGD

SEQ ID NO: 8 - protein, 4 aa, synthetic construct
EIGD

SEQ ID NO: 9 - protein, 4 aa, synthetic construct
ERGD

SEQ ID NO: 10 - protein, 4 aa, synthetic construct
KEGD

SEQ ID NO: 11 - protein, 4 aa, synthetic construct
KLGD

SEQ ID NO: 12 - protein, 4 aa, synthetic construct
KAGD

SEQ ID NO: 13 - protein, 4 aa, synthetic construct
KIGD

SEQ ID NO: 14 - protein, 4 aa, synthetic construct
KRGD

SEQ ID NO: 15 - protein, 4 aa, synthetic construct
LEGD

SEQ ID NO: 16 - protein, 4 aa, synthetic construct
LKGD

SEQ ID NO: 17 - protein, 4 aa, synthetic construct
LAGD

SEQ ID NO: 18 - protein, 4 aa, synthetic construct
LIGD

SEQ ID NO: 19 - protein, 4 aa, synthetic construct
LRGD

SEQ ID NO: 20 - protein, 4 aa, synthetic construct
IEGD

SEQ ID NO: 21 - protein, 4 aa, synthetic construct
IKGD

SEQ ID NO: 22 - protein, 4 aa, synthetic construct
ILGD

SEQ ID NO: 23 - protein, 4 aa, synthetic construct
IAGD

SEQ ID NO: 24 - protein, 4 aa, synthetic construct
IRGD

SEQ ID NO: 25 - protein, 4 aa, synthetic construct
REGD

SEQ ID NO: 26 - protein, 4 aa, synthetic construct
RKGD

SEQ ID NO: 27 - protein, 4 aa, synthetic construct
RLGD

SEQ ID NO: 28 - protein, 4 aa, synthetic construct
RAGD

SEQ ID NO: 29 - protein, 4 aa, synthetic construct
RIGD

SEQ ID NO: 30 - protein, 4 aa, synthetic construct
AEGD

SEQ ID NO: 31 - protein, 4 aa, synthetic construct
AKGD

SEQ ID NO: 32 - protein, 4 aa, synthetic construct
ALGD

SEQ ID NO: 33 - protein, 4 aa, synthetic construct
AIGD

SEQ ID NO: 34 - protein, 4 aa, synthetic construct
ARGD

SEQ ID NO: 35 - protein, 4 aa, synthetic construct
LSVD

SEQ ID NO: 36 - protein, 4 aa, synthetic construct
LSVP

SEQ ID NO: 37 - protein, 4 aa, synthetic construct
LSVG

SEQ ID NO: 38 - protein, 4 aa, synthetic construct
LSDV

SEQ ID NO: 39 - protein, 4 aa, synthetic construct
LSDP

SEQ ID NO: 40 - protein, 4 aa, synthetic construct
LSDG

SEQ ID NO: 41 - protein, 4 aa, synthetic construct
LSPV

SEQ ID NO: 42 - protein, 4 aa, synthetic construct
LSPD

SEQ ID NO: 43 - protein, 4 aa, synthetic construct
LSPG

SEQ ID NO: 44 - protein, 4 aa, synthetic construct
LSGV

SEQ ID NO: 45 - protein, 4 aa, synthetic construct
LSGD

SEQ ID NO: 46 - protein, 4 aa, synthetic construct
LSGP

SEQ ID NO: 47 - protein, 4 aa, synthetic construct
GPKG

SEQ ID NO: 48 - protein, 4 aa, synthetic construct
GPEG

SEQ ID NO: 49 - protein, 4 aa, synthetic construct
GPSG

SEQ ID NO: 50 - protein, 5 aa, synthetic construct
KTTKS

SEQ ID NO: 51 - protein, 5 aa, synthetic construct
RDKYV

(19) United States
(12) Patent Application Publication
(10) Pub. No.: US 2025/0161200 A1
(43) Pub. Date: May 22, 2025

(54) COSMETIC COMPOSITION COMPRISING EXTRACT OF OPHIOPOGON JAPONICUS AND AT LEAST ONE CERAMIDE

(71) Applicant: THE BOOTS COMPANY PLC, Nottingham (GB)

(72) Inventors: Mark Johnson, Nottingham (GB); Michael David Bell, Alfreton, Derbyshire (GB); Victoria Lynne Newton, Beeston, Nottingham (GB); Clare Helena O'Connor, Derbyshire (GB); Mark O'Mahony, West Bridgford, Nottingham (GB)

(21) Appl. No.: 18/839,487

(22) PCT Filed: Feb. 16, 2023
(86) PCT No.: PCT/EP2023/025071

§ 371 Date: Aug. 19, 2024

(30) Foreign Application Priority Data
Feb. 18, 2022 (EP) 22020066.1

(51) Int. Cl. A61K 8/9794; A61K 8/67; A61K 8/68; A61Q 17/04; A61Q 19/08; A61Q 19/10

(52) U.S. Cl. CPC A61K 8/9794; A61K 8/675; A61K 8/68; A61Q 17/04; A61Q 19/08; A61Q 19/10; A61K 2800/592

(57) ABSTRACT

According to the present invention there is provided a cosmetic composition suitable for use in application to menopausal skin comprising extract of Ophiopogon japonicus and at least one ceramide.

### CROSS-REFERENCE TO RELATED APPLICATIONS

This application is a U.S. national stage of International Application No. PCT/EP2023/025071, filed Feb. 16, 2023, which claims priority to European Patent Application No. 22020066.1, filed Feb. 18, 2022.

### INCORPORATION BY REFERENCE OF MATERIAL SUBMITTED ELECTRONICALLY

The Sequence Listing, which is a part of the present disclosure, is submitted concurrently with the specification as an XML file. The name of the XML file containing the Sequence Listing is “70751_SeqListing.xml”, which was created on Aug. 15, 2024, and is 44,847 bytes in size. The subject matter of the Sequence Listing is incorporated herein in its entirety by reference.

### TECHNICAL FIELD

The present invention relates to a composition specifically designed for application to menopausal skin.

### BACKGROUND TO THE INVENTION

Cosmetically, healthy looking skin is characterised by skin that is well moisturized, smooth and blemish free, luminous and radiant, even in skin tone and non-oily. Skin epidermis undergoes a constant renewal process wherein the maintenance of tissue integrity is controlled by sustaining a constant epidermal cell pool in which the rate of mitosis and the rate of desquamation and apoptosis are balanced. Constant renewal of the skin layers maintains the integrity of the skin's barrier function. This is known as epidermal homeostasis. Throughout life, transient changes in for example lifestyle, mental well-being, sleep, nutrition, environmental exposure can lead to transient fluctuations in skin appearance driven primarily by an impact on epidermal homeostasis. Hormones represent an additional exposomal factor, whether produced naturally by endocrine glands coinciding with life stage or whether provided by supplementation as in contraception, performance enhancers and hormone replacement therapy (HRT) and there is growing evidence that hormones can impact on dermatologic mechanisms that will have an impact on cosmetic healthy skin concerns.

The most well researched hormonal skin condition is acne. This skin disease involves a complex cascade of events that are controlled by androgen hormones impacting on the pilosebaceous unit leading to increased sebum production and resulting inflammation. Fluctuating sex hormones are also implicated in pre-menstrual “acne” flares with peak testosterone levels associated with highest acne prevalence (Williams M, Cunliffe W J. Explanation for premenstrual acne. Lancet. 1973; 2(7837):1055-1057 and Geller, Rosen, Frankel, Goldenberg. Perimenstrual Flare of Adult Acne. J Clin Aesthet Dermatol. 2014 August; 7(8): 30-34.). However, the impact of hormone fluctuations is not limited to acne.

Probably the most researched hormone and its impact on the skin is oestrogen. The menopause represents a transition period when oestrogen levels fall precipitously. The skin shows the effects of depleted oestrogen with reported skin dryness, accelerated worsening of wrinkles and a loss of firmness being key patient and consumer concerns. These concerns correspond with changes in structural and architectural components (such as collagen abundance) as well as decreased sebum production impacting the quality of the skin barrier function. The outermost layer of the skin, the stratum corneum, is principally responsible for the skin's role as a barrier to environmental exposures. It is formed from skin cells that start off as keratinocytes in the epidermal basal layer before they journey up through the epidermal layers, differentiating by flattening and becoming keratin rich before finally becoming corneocytes in the stratum corneum. Various proteins including enzymes are involved in this differentiation process. The enzyme transglutaminase for example catalyses the cross linking of proteins such as involucrin and loricrin that are key components of the resulting corneocyte cell envelop. Keratinocytes possess oestrogen receptors which are believed important regulators of cell differentiation, proliferation and epithelialisation and therefore epidermal homeostasis. The observed changes in skin described above in post-menopausal skin are partially rescued in HRT cohorts, suggesting an important role for oestrogen in epidermal homeostasis. It is therefore not surprising that the rapid decline in oestrogen following the menopause can lead to a disruption to the essential epidermal processes such as differentiation and ultimately lead to an impairment in the skin barrier function. These mechanisms are reasonably believed to be behind the visible appearance changes observed in peri- and post-menopausal skin. Hence, although without wishing to be limited to theory, it is believed that the depletion of oestrogen leads to dysregulation of the overall skin barrier function and epidermal homeostasis resulting to noticeable changes in the appearance of skin such as dryness, elasticity, texture, tone, radiance and luminosity.

In addition to the above, psychological stress is also known to exacerbate various skin diseases including psoriasis, atopic dermatitis and acne. During periods of stress the human body makes and releases the hormone cortisol, commonly referred to as the ‘Stress Hormone’. Heightened cortisol levels have also been associated with increased inflammation which can in turn can impact epidermal homeostasis and skin barrier function. In studies, final exam and interview stress have been shown to impact skin barrier function, the latter associated with increased plasma cortisol levels and upregulated inflammation (Effect of “Rose Essential Oil” Inhalation on Stress-Induced Skin-Barrier Disruption in Rats and Humans. Mika Fukada, Eri Kano, Michio Miyoshi, Ryoichi Komaki, Tatsuo Watanabe. Chemical Senses, Volume 37, Issue 4, May 2012, Pages 347-356). It is not surprising therefore that skin is a known target for cortisol, indeed the skin is also able to produce its own corticosteroids, with corticosteroid (CR) receptors available in the skin to mediate inflammatory biological effects.

The present application seeks to address some of the concerns of women experience a change in skin health, moisturization and appearance during the peri- and post-menopausal period of their lives.

### SUMMARY OF THE INVENTION

According to the present invention there is provided a cosmetic composition suitable for use in application to menopausal skin comprising extract of Ophiopogon japonicus and at least one ceramide.

### DESCRIPTION OF THE INVENTION

The present invention relates to cosmetic composition suitable for use in application to peri- and post-menopausal skin and comprise extract of Ophiopogon japonicus and at least one ceramide. Unless otherwise indicated, a reference to the composition of the invention is a reference to the compositions of all aspects of the invention.

Extract of Ophiopogon japonicus

The composition of the present invention comprises extract of Ophiopogon japonicus. Preferably the raw material ingredient used in the present invention is an extract of the root of the Ophiopogon japonicus plant.

Extract of Ophiopogon japonicus root comprise a steroidal glycoside known as Ophiopogonin D (OP-D) which provides anti-inflammatory benefits. Ophiopogon root also contains various additional biologically active compounds, including steroidal saponins, homoisoflavonoids, and polysaccharides, which possess anti-oxidative, anticancer, antidiabetic, immunomodulatory, anti-acute inflammatory, antitussive, and antimicrobial activities.

Extract of Ophiopogon japonicus root is available from Silab and sold under the trade names Cohesium and Ad-Resyl.

Extract of Ophiopogon is present in the compositions of the present invention at a level of at least 0.01%, at least 0.10%, at least 1.0%. Extract of Ophiopogon is present in the compositions of the present invention at a level of less than 10%, less than 7.5%, less than 5.0%. Extract of Ophiopogon is present in the compositions of the present invention at a level of from 0.1 to 10%, 0.75% to 7.5% and preferably 1 to 5%.

### Ceramide

Ceramides are a family of waxy lipid molecules. A ceramide is composed of sphingosine and a fatty acid. In an optional embodiment of the present invention, the composition comprises at least one ceramide. Whilst not be limited by theory, said ceramide may be selected from the group consisting of ceramide 1 (ceramide EOP), ceramide 2 (ceramide NG), ceramide 3 (ceramide NP) and/or ceramide 6 (ceramide AP). The above ceramides are well known in the art and defined by this nomenclature. However, for the avoidance of doubt: ceramide 1 (ceramide EOS)—comprises a sphingosine base and a non-hydroxy fatty acid chain linked to linoleic acid; ceramide 2 (ceramide NS) comprises a sphingosine base and a non-hydroxy fatty acid chain; ceramide 3 (ceramide NP) comprises a phytosphingosine base (OH group at the C4 position on the sphingosine base molecule) and a non-hydroxy fatty acid chain ceramide 6 (ceramide AP) comprises a phytosphingosine base (OH group at the C4 position on the sphingosine base molecule) and an alpha-hydroxy fatty acid chain (additional OH group on the fatty acid v.s. non-hydroxy).

In a preferred aspect of the present invention, the composition preferably comprises ceramide 3 (ceramide NP), also known as N-oleoyl-4-hydroxysphinganine. In a further preferred but optional aspect of the present invention, the composition preferably comprises ceramide 6 (ceramide AP).

Alternatively, the composition may comprise at least two ceramides, such as ceramide 3 and ceramide 6. Alternatively, the composition may comprise at least 3 ceramides, such as ceramide 1, ceramide 3, and ceramide 6.

In one embodiment of the present invention the ceramide is Skinmimic (trade name) commercially available from Evonik. Skinmimic is an ingredient comprising a number of ceramides including Ceramide EOP, Ceramide EOS, Ceramide NP, Ceramide NS, Ceramide AP, Caprooyl Phytosphingosine and Caprooyl Sphingosine.

Where present, ceramide (e.g. singularly or combined amounts of ceramides) may be present in the composition in an amount by weight of at least 0.003%, at least 0.005%, at least 0.008% or at least 0.01%; and/or the at least one ceramide (e.g. combined amounts of ceramides 1, 3 and 6) may be present in the composition in an amount by weight of 0.1% or less, 0.05% or less, 0.03% or less, or 0.01% or less.

Where present ceramide (e.g. singularly or combined amounts of ceramides) may be present in the composition in an amount by weight of from 0.003% to 0.03% or from 0.005% to 0.02%.

### Phytoestrogenic Isoflavone

Phytoestrogen is an oestrogen-like compounds found in plants which is structurally similar. The composition of the present invention optionally, but preferably comprises a phytoestrogenic isoflavone derived from a phytoestrogenic plant. Phytoestrogenic plants include soyabean (Glycine max), red clover (Trifolium pratense), white clover (Trifolium repens), alfalfa (Medicago sativa), garlic (Allium sativum), celery (Apiaceae graveolens), carrot (Daucus carota), potato (Solanum tuberosum), rice (Oryza sativa), wheat (Triticum aestivum), sweet potato (Ipomoea batatas), apple (Malus domestica), pomegranate (Punica granatum), chaste berries (Vitex agnus-castus), coffee (Coffea arabica and Coffea robusta) and mixtures thereof.

In one embodiment the phytogenic isoflavone where present is selected from phytogenic plants selected from the group consisting of soyabean (Glycine max), red clover (Trifolium pratense), white clover (Trifolium repens), alfalfa (Medicago sativa) and mixture thereof.

Isoflavones can be classified into the following two types depending on the presence or absence of glucoside: (1) glycosylated phytoestogenic isoflavones (also referred to as phytoestogenic isoflavone glycosides) that include daidzein, genistein, glycitin, malonyldaidzein, malonylgenistein, malonylglycitin, acetyldaidzin and acetylglycitin; and (2) deglycosylated phytoestogenic isoflavones (also referred to as phytoestogenic isoflavone aglycones) that include daidzein, genistein and glycitein. Deglycosylated phytoestogenic isoflavones have higher bioavailability as compared to glycosylated phytoestogenic isoflavones, and thus may achieve better health benefits.

Preferred isoflavones are selected from the group consisting of genistein (5, 7, 4′-Trihydroxy-Isoflavon), genistein (5, 7,4′-Trihydroxy-isoflavone-7-glucoside), daidzein (7.4′-Dihydroxy-Isoflavone), equol (4′, 7-Dihydroxyisoflavan), daidzein (7.4′-Dihydroxy-isoflavone-7-glucoside), biochanin A (5.7-Dihydroxy-4′-of-methoxy-Isofla), glycitein (7.4′-dihydroxy-6-methoxy-Isoflavon), glycitin (7.4′-Dihydroxy-6-methoxy-isoflavone-7-glucoside), orobol (5.3′, 4′-Trihydroxy-7-methoxy-Isoflavone), O-robol (5, 7, 3′, 4′-Tetrahydroxy-Isoflavone), santal (5, 7, 3′-Trihydroxy-4-methoxy-Isoflavone) and/or enzyme inhibitors (5.4′-Dihydroxy-7-methoxy-Isoflavone) and mixtures thereof.

In one embodiment of the present invention the phytogenic isoflavone where present is a soy isoflavone. The soyabean is known to contain a phytoestrogen, soy isoflavone, which is able to bind to oestrogen receptors with capability to produce, albeit weak, oestrogen-similar physiological effects on the female body. The literature on the subject suggests that soy isoflavone may be useful for women in peri- and post-menopause in treatment of menopausal symptoms such as mood swings, temperature fluctuations, and fatigue. In the main, soy isoflavones are described with reference to a foodstuff or supplement and are ingested.

Methods of preparing soy isoflavones are known in the art and soy isoflavones can be readily bought from manufacturers. Essentially, traditional soymilk (also known as filtered soymilk) is prepared by pulverizing a whole soybean material soaked in water, subsequently optionally filtering the resulting soybean slurry with gauze, and optionally heating the thus obtained filtrate for sterilization. Although the filtration treatment can remove soybean dregs having a large particle size from the soybean slurry, such treatment also reduces the amount of active ingredients of the soybean slurry, thereby reducing the value of the filtered soymilk.

In one embodiment of the present invention the phytogenic isoflavone where present is contained within a liposome structure. Liposomal delivery system improves are preferred for the improved penetration of the isoflavone across the epidermis.

In one embodiment of the present invention the phytogenic isoflavone is Lipobelle (trade name) commercially available from Mibelle Biochemistry Group. Lipobelle is a liposomal preparation of genistein.

In one embodiment of the present invention the cosmetic or dermatological preparations according to the invention may comprise 0.001 to 20% by weight, particularly preferably 0.01 to 5% by weight, of phytogenic isoflavone.

In one embodiment, the extract of Ophiopogon japonicus and phytogenic isoflavone are present at a ratio of from 5:1 to 1:2. In one embodiment, the extract of Ophiopogon japonicus and phytogenic isoflavone are present at a ratio of from 4:1 to 1:1. In one embodiment, the extract of Ophiopogon japonicus and phytogenic isoflavone are present at a ratio of from 3.5:1 to 2:1.

### Vitamin B3 or Vitamin B3 Derivative

The cosmetic compositions of the present invention preferably comprise Vitamin B3, also known as niacin or nicotinic acid, and/or vitamin B3 derivative. The physiologically active form of niacin is niacinamide. Niacin and niacinamide (nicotinamide or nicotinic acid amide) function in the body as a component of two coenzymes: nicotinamide adenine dinucleotide (NAD) and nicotinamide adenine dinucleotide phosphate (NADP).

The compositions used in the present invention may comprise a safe and effective amount of a (natural or synthetic) vitamin B3 compound. As used herein, “vitamin B3 compound” means a compound having the formula:

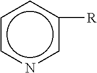

wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B3 compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

Suitable esters of nicotinic acid include nicotinic acid esters of C1-C22, preferably C1-C16, more preferably C1-C6 alcohols. The alcohol may be straight-chain or branched chain, cyclic or acyclic, saturated or unsaturated (including aromatic), and substituted or unsubstituted. Preferred esters are those which do not yield a visible flushing response after application to the skin. Alternatively, a nicotinic acid material which does produce a flushing response can be used, if used at a lower dose to reduce the flushing effect. Non-flushing esters of nicotinic acid include tocopherol nicotinate and inositol hexanicotinate. Tocopherol nicotinate is preferred.

Other derivatives of the vitamin B3 compound are derivatives of niacinamide resulting from substitution of one or more of the amide group hydrogens. Non-limiting examples of derivatives of niacinamide useful herein include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid and nicotinyl hydroxamic acid.

Exemplary nicotinyl alcohol esters include nicotinyl alcohol esters of carboxylic acids salicylic acid, acetic acid, glycolic acid, palmitic acid and the like. Other suitable vitamin B3 compounds are selected from the group consisting of 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine and mixtures thereof.

Examples of the above vitamin B3 compounds are well known in the art and are commercially available from a number of sources, e.g., the Sigma Chemical Company (St. Louis, MO); ICN Biomedicals, Inc. (Irvin, CA) and Aldrich Chemical Company (Milwaukee, WI).

The compositions of the present invention preferably comprise one or more vitamin B3 compounds. Preferred vitamin B compounds are selected from the group consisting of C1-C22 straight-chain or branched chain, cyclic or acyclic, saturated or unsaturated (including aromatic), and substituted or unsubstituted esters of nicotinic acid, nicotinyl amino acids, nicotinuric acid, nicotinyl hydroxamic acid, nicotinyl alcohol esters of carboxylic acids including salicylic acid, acetic acid, glycolic acid, palmitic acid, 2-chloronicotinamide, 6-aminonicotinamide, 6-methylnicotinamide, n-methylnicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine, niacinamide, tocopherol nicotinate and mixtures thereof.

Preferred vitamin B3 compounds are niacinamide and tocopherol nicotinate. Niacinamide is more preferred.

Salts of the vitamin B3 compound are also useful herein. Non-limiting examples of salts of the vitamin B3 compound useful herein include organic or inorganic salts, such as inorganic salts with anionic inorganic species (e.g., chloride, bromide, iodide, carbonate, preferably chloride), and organic carboxylic acid salts (including mono-, di- and tri-C1-C18 carboxylic acid salts, e.g., acetate, salicylate, glycolate, lactate, malate, citrate, preferably monocarboxylic acid salts such as acetate).

The vitamin B3 compound may be included as the substantially pure material, or as an extract obtained by suitable physical and/or chemical isolation from natural sources. The vitamin B3 compound is preferably substantially pure, more preferably essentially pure.

Vitamin B or vitamin B derivative may be present in the composition in an amount by weight of at least 0.01%, at least 0.05%, at least 0.1%, at least 0.5%. The vitamin B or vitamin B derivative may be present in the composition in an amount by weight of 5% or less, 4% or less, 3% or less, 2% or less, 1% or less, 0.5% or less, 0.1% or less, or 0.05% or less. Where more than one vitamin B or vitamin B derivative is present, the values apply to the total amount of vitamin B or vitamin B derivative in the composition.

In particular, the vitamin B or vitamin B derivative (e.g. niacinamide) may be present in the composition in an amount by weight of from 0.01 to 5%, 0.05% to 3%, from 0.1% to 3%, 0.1% to 2%, from 0.5% to 1%, or from 0.5% to 2%.

### Skin Soothing Agents

The composition of the present invention may comprise a skin soothing agent. Said skin soothing agents have the effect of calming irritated or sensitive skin and hence have anti-inflammatory activity. Such ingredients are often, but not exclusively, derived from plants.

A preferred skin soothing agent is extract of Centella asiatica. Extract of Centella asiatica, also known as Gotu kola or India Pennyworth. Preferably the raw material known as Centella asiatica is an extract from the leaf of the plant and is characterized by the presence of biologically active triterpenes, including madecassoside, and a polyphenolic fraction contributing to the biological efficacy of the extract. This product is known to provide antioxidant benefits and to promote collagen type I and III synthesis. This extract is commercially available under the trade name Centevita from Indena.

Extract of Centella asiatica, meaning the extract acquired from a supplier, may be present in the compositions of the present invention at a level of from 0.001%, alternatively from 0.002%, alternatively from 0.1% by weight of the composition. Extract of Centella asiatica may be present in the compositions of the present invention at levels of 0.5% or less, alternatively less than 0.25% or less, or alternatively 0.1% or less. In one embodiment extract of Centella asiatica may be present in the composition of the present invention at a level of from 0.001% to 0.5% by weight of the composition. In one embodiment extract of Centella asiatica may be present in the composition of the present invention at a level of from 0.005% to 0.25% by weight of the composition. In one embodiment extract of Centella asiatica may be present in the composition of the present invention at a level of from 0.01% to 0.1% by weight of the composition.

An alternative skin soothing agent is monocyclic sesquiterpene alcohol. Sesquiterpenes are a class of terpenes that consist of three isoprene units and often have the molecular formula C₁₅H₂₄. Sesquiterpenes may be acyclic or contain rings. Alternatively, the skin soothing agent may be an alcohol derivative of a monocyclic (single ring) sesquiterpene. For example, the monocyclic sesquiterpene alcohol may be α-bisabolol (shown below, left) or β-bisabolol (shown below, right). Synthetic bisabolol is usually a racemic mixture: α-(±)-bisabolol

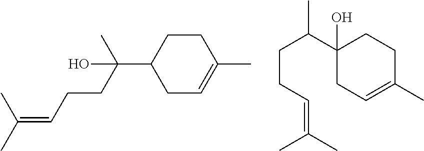

The monocyclic sesquiterpene alcohol (e.g. α-bisabolol) may be present in the composition in an amount by weight of at least 0.001%, at least 0.005%, at least 0.01%, at least 0.05%, at least 0.1%, at least 2.5%; and/or the monocyclic sesquiterpene alcohol (e.g. α-bisabolol) may be present in the composition in an amount by weight of 2% or less, 1% or less, 0.5% or less.

In particular, the monocyclic sesquiterpene alcohol (e.g. α-bisabolol) may be present in the composition in an amount by weight of from 0.001% to 2%, from 0.01% to 1%, or from 0.05% to 0.5%.

### Other Components

The compositions of the present invention may be aqueous or emulsion, including water-in-oil or oil-in-water emulsions. Where the composition is aqueous, it preferably comprises from 10% to 99.9% by weight water. In a preferred embodiment, aqueous compositions comprise from 20% to 80% by weight water. In a preferred embodiment, aqueous compositions comprise from 40% to 70% by weight water. The water phase also preferably contents humectants which can be chosen from the following list of diols or triols. Preferred diols are selected from the group consisting of; pentylene glycol, caprylyl glycol, butylene glycol, di-propylene glycol, ethylhexylglycerine, propanediol, hexenediol, glycerol, butylene glycol, propylene glycol, isoprene glycol, dipropylene glycol, pentylene glycol, hexylene glycol, polypropylene glycol, butylene glycol, polyethylene glycol, sorbitol, glucitol, mannitol, hydroxypropyl sorbitol, erythritol, threitol, pentaerythritol, xylitol, and mixtures thereof. Preferred triols are selected from the group consisting of: hexanetriol, glycerine, ethoxylated glycerin, propoxylated glycerin, and mixtures thereof.

Where the composition is an emulsion, the oil phase of an emulsion can be provided by any suitable oily component. Suitable oils for the oil phase may comprise for example: a) hydrocarbon oils, such as paraffin or mineral oils; b) waxes, such as beeswax or paraffin wax; c) natural oils, such as sunflower oil, apricot kernel oil, shea butter or jojoba oil; d) silicone oils, such as dimethicone, silicone elastomer, cyclomethicone or cetylidimethicone; e) fatty acid esters and ethers, such as isopropyl palmitate, isopropyl myristate, Caprylic/capric triglyceride, C12-15 alkyl benzoate and polypropylene glycol-15 stearyl ether; f) fatty alcohols, such as cetyl alcohol or stearyl alcohol, g) branched chain primary alcohols such as octyldodecanol; or h) mixtures thereof, for example, the blend of waxes available commercially under the trade name Cutina (BASF). Preferably, the emulsion comprises 0.1% to 55%, more preferably from 15% to 50%, most preferably from 30% to 45% by weight of the emulsion, of oil phase. Preferably the oil phase of the emulsion comprises oil at a level between 50% and 99.9% by weight of the oil phase. More preferably the oil phase comprises oil at a level of from 60% to 99.9%, more preferably from 70% to 99.9%, and even more preferably from 80% to 99.9% by weight of the oil phase.

Preferably the oil phase of the water-in-oil emulsion comprises a silicone oil. Where present, the silicone-containing oil phase preferably comprises an organopolysiloxane oil. The organopolysiloxane oil for use in the composition may be volatile, non-volatile, or a mixture of volatile and non-volatile silicones. The term “nonvolatile” as used in this context refers to those silicones that are liquid or gel under ambient conditions and have a flash point (under one atmospheric of pressure) of greater than 100° C. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes.

Preferred for use herein are organopolysiloxanes selected from polyalkylsiloxanes, alkyl substituted dimethicones, cyclomethicones, trimethylsiloxysilicates. dimethiconols, polyalkylaryl siloxanes, and mixtures thereof. More preferred for use herein are polyalkylsiloxanes and cyclomethicones. Preferred among the polyalkylsiloxanes are dimethicones.

Optionally a suitable silicone is a silicone elastomer. Suitable for use herein are silicone elastomers which can be emulsifying or non-emulsifying crosslinked siloxane elastomers or mixtures thereof. No specific restriction exists as to the type of curable organopolysiloxane composition that can serve as starting material for the crosslinked organopolysiloxane elastomer. Examples in this respect are addition reaction-curing organopolysiloxane compositions which cure under platinum metal catalysis by the addition reaction between SiH-containing diorganopolysiloxane and organopolysiloxane having silicon-bonded vinyl groups; condensation-curing organopolysiloxane compositions which cure in the presence of an organotin compound by a dehydrogenation reaction between hydroxyl-terminated diorganopolysiloxane and SiH-containing diorganopolysiloxane and condensation-curing organopolysiloxane compositions which cure in the presence of an organotin compound or a titanate ester.

Preferably the oil phase comprises silicone, and most preferably, a silicone elastomer. Preferably, the emulsion composition includes from 20% to 35%, by weight of the emulsion composition, of the silicone elastomer raw material.

The water-in-oil emulsions of the present invention preferably comprise an emulsifier. In a preferred embodiment, the composition comprises from 0.1% to 10% emulsifier, more preferably from 0.25% to 7.5%, still more preferably from 0.5% to 5%, emulsifier by weight of the composition. The emulsifier helps disperse and suspend the aqueous water phase within the preferred silicone oil phase.

### Saccharide Isomerate

The composition may comprise saccharide isomerate. Saccharide isomerate consists mainly of glucose, fructose mannose and galactose.

Saccharide isomerate mimics NMF (the Natural Moisturising Factor) found in human skin and is derived from plant based glucose. Saccharide isomerate is commercially available under the trademark name PENTAVITIN® from DSM Nutritional Products Ltd.

The saccharide isomerate may be present in the composition in an amount by weight of at least at least 0.05%, at least 0.1%, at least 0.2% or at least 0.3%; and/or the saccharide isomerate may be present in the composition in an amount by weight of 2% or less, 1% or less, 0.8% or less, 0.6% or less or 0.4% or less.

In particular, the saccharide isomerate may be present in the composition in an amount by weight of from 0.1% to 0.9% or from 0.2% to 0.6%.

### Antioxidant Agent

The compositions of the present invention may optionally comprise an antioxidant agent. Suitable antioxidant agents may include: a) ascorbic acid its salts, esters, glucosides and glucosamines, particularly sodium ascorbyl phosphate, magnesium ascorbyl phosphate and ascorbyl palmitate b) vitamin E (tocopherol) and its esters, particularly tocopheryl acetate, as well as Dimethyl methoxy chromanol which is a synthetic analogue of gamma tocopherol, available from Lipotec S.A. polygon Industrial Camri Ral, under the tradename Lipochroman-6 c) herbal extracts, particularly gingko biloba, such as that available under the trade name “gingko biloba Leaf Powder” from Univar PLC, Morus alba, such as that available under the trade name “Mulberry Concentrate” from Solabia, Origanum vulgare, such as that available under the trade name “Pronalen Origanum HSC” from S Black Ltd, Panax ginseng, such as that available under the trade name “Panax ginseng 1.1 extract 4294” from S Black Ltd or “Phytexcell Panax ginseng” available from Croda Chemicals Ltd, birch extract such as those available from Cosmetochem (U. K.) Ltd under the trade names “Super Herbasol Extract Birch” and “HP Herbasol Betula” and those available from Blagden Chemicals under the tradenames “Phytelene of Birch” and “Aqueous Spray Dried Birch”, Camellia sinensis, such as that available under the trade name “Herbal Extract Green Tea 75% Solids” from Nichimen Europe, Rosmarinus officinalis, such as that available under the trade name “Pronalen Rosemary” from S. Black, Acerola cherry powder, such as that available as Acerola PE from Gee Lawson, Emblica extract sold under the tradename Emblica™ by Merck Specialty chemicals, and Grape Seed oil, such as that available from Chesham Chemicals Limited.

The source of the antioxidant activity in some of these agents is often not fully understood; for example, it is believed that the antioxidant activity of Ginkgo biloba extract arises from the presence of flavonglycocides and/or terpenelactones which may be free-radical inhibitors. Birch extract may be produced by extracting the dried leaves of Betula alba with a suitable solvent. It is believed that the anti-free radical activity of birch extract arises due to the presence of flavonoids such as hyperosid, quencitrosid and/or myricetol-3digalactosid which may be free-radical inhibitors. Such products are then often sold as mixtures or solutions.

Thus the antioxidant agent may consist of a number of active ingredients which are free-radical inhibitors or may also comprise suitable diluents and/or carriers (such as when the anti-free radical agent is some of the products mentioned herein). Thus there may be some confusion as to the actual level of agent within a commercially available product. Accordingly, the amounts of antioxidant agents used in the present invention are expressed as dry weights, as understood by a man skilled in the art. The total amount of antioxidant agents present in the composition may range from 0.005% to 10% by weight, preferably 0.5% to 5%, most preferably 1% to 3.5% by weight of the composition.

Particularly preferred synergistic combinations of antioxidant agents suitable for inclusion in a skin care composition of the present invention are: Panax ginseng, Morus alba and magnesium ascorbyl phosphate; Panax ginseng, Morus alba and sodium ascorbyl phosphate; Panax ginseng, Morus alba and Rosmarinus officinalis; Panax ginseng, Morus alba and Origanum vulgare.

In these preferred combinations (a) the Panax ginseng is preferably present in an amount of 0.005% to 0.10%, more preferably 0.01% to 0.05% by weight of the composition; (b) the Morus alba is preferably present in an amount of 0.0005% to 0.01%, more preferably 0.001% to 0.005% by weight of the composition; (c) the sodium or magnesium ascorbyl phosphate is preferably present in an amount of 0.05% to 2.5%, preferably 0.1% to 2%, most preferably 0.15% to 1.5% by weight of the composition and (d) the Rosmarinus officinalis or Origanum vulgare is preferably present in an amount of 0.01% to 0.5%, more preferably 0.05% to 0.2% by weight of the composition.

### Emulsifiers

The composition of the present invention may comprise an emulsifier. Suitable emulsifiers include all those suitable for the purpose and known by those skilled in the art for use in skin care products. Preferably these emulsifiers have an HLB value of or above 5, more preferably from 5 to 17, and still more preferably from 10 to 17.

Among the emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Nonlimiting preferred examples of these non-silicon-comprising emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

### Peptides

The compositions of the present invention may comprise further peptides. Preferably said additional peptides are selected from the group consisting of dipeptides, tripeptides, tetrapeptides, pentapeptides and mixtures thereof. By tripeptides, it is meant compound comprising an uninterrupted sequence of three amino acids. By tetrapeptides, it is meant a compound comprising an uninterrupted sequence of four amino acids. By pentapeptide it is meant a compound comprising an uninterrupted sequence of five amino acids.

### Dipeptides:

The compositions of the present invention may comprise a dipeptide selected from the group consisting of acetyl dipeptide 1 cetyl ester (Tyrosine-Arginine; commercially available under the trade name Idealift from Sederma), acetyl dipeptide 3 aminohexanoate (Alanine-Arginine), azelaoyl bisdipeptide 10 (Alanine-Histidine), coumaroyl dipeptide 3 (Alanine-Arginine), dicetyl dipeptide 9 (Glutamic acid-Lysine), dipeptide diamino butyroyl benzylamide diacetate, dipeptide 1 (Tyrosine-Arginine), dipeptide 10 (Alanine-Histidine), dipeptide 11 (Cysteine-Lysine), dipeptide 12 (Lysine-Lysine), dipeptide 14 (Alanine-Threonine), (dipeptide 15 (Glycine-Glycine), dipeptide 16 (Leucine-Leucine), dipeptide 17 (Glycine-Proline), dipeptide 18 (Glycine-Histidine), dipeptide 19 (Leucine-Glutamic acid), dipeptide 2 (Valine-Tryptophan), dipeptide 20 (Lysine-Methionine), dipeptide 3 (Alanine-Arginine), dipeptide 4 (Cysteine-Glycine), dipeptide 5 (Lysine-Valine), dipeptide 6 (Proline-Hydroxyproline), dipeptide 7 (Lysine-Threonine), dipeptide 8 (Alanine-Hydroxyproline), dipeptide 8 HCL (Alanine-Hydroxyproline), dipeptide 9 (Glutamic acid-Lysine), hexanoyl dipeptide 3 norleucine acetate (Alanine-Arginine), methyl undecylenoyl dipeptide 16 (Leucine-Leucine), nicotinoyl dipeptide 22 (Serine-Valine), nicotinoyl dipeptide 23 (Proline-Serine), nicotinoyl dipeptide 24 (Glutamic acid-Threonine), nicotinoyl dipeptide 26 (Arginine-Valine), oleoyl dipeptide 15 (Glycine-Glycine), palmitoyl dipeptide 10 (Alanine-Histidine), palmitoyl dipeptide 13 (Tryptophan and Glutamic acid), palmitoyl dipeptide 17 (Glycine-Proline), palmitoyl dipeptide 5 diaminobutyroyl hydroxythreonine (Lysine-Valine), palmitoyl dipeptide 5 diaminohydroxybutyrate (lysine-Valine), palmitoyl dipeptide 7 (Lysine-Threonine) and mixtures thereof.

Dipeptides where present are preferably incorporated into the emulsion of the present invention at a level of from 0.1 to 50000 ppm, more preferably from 1 to 5000 ppm, most preferably from 10 to 500 ppm.

### Tripeptides:

The compositions of the present invention preferably comprise a tripeptide. Said tripeptide may be naturally occurring or of synthetic origin. Suitable tripeptides include tripeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, derivatives thereof and mixtures thereof.

Particularly preferred tripeptides comprise one or more His-based tripeptides. However another suitable tripeptide may be Arg-Lys-Arg. Particularly preferred tripeptides are based on the structure Gly-His-Lys and its analogs and derivatives thereof. These are collectively known herein as GHK-tripeptides. Indeed, the preferred tripeptide in accordance with this aspect of the invention has this exact sequence of amino acids. Analogs of the preferred tripeptide useful herein include those in which one or more of the three amino acids are reorganized or rearranged within the sequence (e.g., Gly-Lys-His) and/or where no more than two amino acids are substituted (e.g., His-Ala-Om). However, most preferably, amino acids substituted for Gly include an aliphatic side chain such as, without limitation, beta-Ala, Ala, Val, Leu, Pro, Sarcosine (Sar) and Ile. Most preferred are Ala, Leu and Ile. The most preferable amino acid substituted for Lys or His include those having a side chain that includes, predominantly, a charged nitrogen at a pH of 6, such as, without limitation, Pro, Lys, Arg, His, Desmosine and Isodesmosine. Most preferably, Lys is replaced with Orn, Arg, or Citrulline.

Derivatives are also considered to be encompassed by the term GHK-tripeptides in accordance with the present invention, (and therefore also the more generic term tripeptides). Derivatives of GHK-tripeptides in accordance with the present invention include derivatives of the substituted and rearranged tripeptides described herein. These derivatives include, inter alia, acyl-derivatives, which are tripeptides substituted with one or more straight-chain or branched-chain, long or short chain, saturated or unsaturated, substituted with a hydroxy, amino, acyl amino, sulfate or sulfide group, or unsubstituted, which can be derived from acetic acid, capric acid, lauric acid, myristic acid, octanoic acid, palmitic acid, stearic acid, behenic acid, linoleic acid, linolenic acid, lipoic acid, oleic acid, isostearic acid, elaidoic acid, 2-ethylhexaneic acid, coconut oil fatty acid, tallow fatty acid, hardened tallow fatty acid, palm kernel oil fatty acid, lanolin fatty acid and the like. Preferable examples of the acyl group include an acetyl group, a palmitoyl group, an elaidoyl group, a myristyl group, a biotinyl group and an octanoyl group. These may be substituted or unsubstituted. When substituted, they are preferably substituted with hydroxyl or sulphur comprising groups such as, without limitation SO₃H, SH or S—S.

His-based tripeptides include at least one Histadine amine acid. The other two amino acids in the sequence may be the same or different. Thus, contemplated are, without limitation, His-Xaa-Xaa, His-Xaa-Xbb, His-Xbb-Xaa, Xbb-His-Xbb, Xbb-His-Xaa, Xaa-His-Xbb, Xaa-Xaa-His, Xaa-Xbb-His, Xbb-Xaa-His and Xbb-Xbb-His, where Xaa and Xbb are two different amino acids, although either can be His. Preferably, at least one of the other amino acids is Gly, beta-Ala, Ala, Val, Leu, Pro, Sarcosine (Sar) or Ile. Preferably, at least one of the other amino acids is Pro, Lys, Arg, His, Desmosine and Isodesmosine. Most preferably, Lys is replaced with Orn, Arg, or Citrulline. Derivatives are also considered to be encompassed by the term His-based tripeptides in accordance with the present invention, (and therefore also the more generic term tripeptides). These derivatives include, inter alia, acyl-derivatives, which are tripeptides substituted with one or more straight-chain or branched-chain, long or short chain, saturated or unsaturated substituted or unsubstituted acyl group(s) having from 1 to 29 carbon atoms. The acyl groups which can be used are the same as those described for the GHK-tripeptides.

Particularly preferred embodiments of tripeptides in accordance with the present invention include N-Acyl-Gly-His-Lys and most preferably, N-Palmitoyl-Gly-His-Lys. Preferred commercially available tripeptide and tripeptide derivative comprising compositions include Biopeptide-CL from SEDERMA, Maxilip® from SEDERMA, Biobustyl® from SEDERMA.

The tripeptides of the present invention are preferably used in amounts that can be as little as 0.10 ppm to 10,000 ppm, preferably between 0.50 ppm to 5,000 ppm, more preferably from 1 ppm to 1000 ppm, and most preferably from 1 ppm to 500 ppm. These are again based on a % w/w basis. Thus 100,000 ppm is 10% by weight of the emulsion.

### Tetrapeptides:

The emulsions of the present invention preferably comprise a tetrapeptide. These may be one or more rigin-based tetrapeptides, one or more ALAMCAT-tetrapeptides or mixtures thereof. These tetrapeptides may be naturally occurring or of synthetic origin. Suitable tetrapeptides for use in the present composition include those selected from the group consisting of tetrapeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 34, 35, derivatives thereof and mixtures thereof.

Rigin-based tetrapeptides in accordance with the present invention are based on the structure Gly-Gln-Pro-Arg (SEQ ID NO:1)(Rigin) and include its analogs and derivatives thereof. Rigin is a preferred tetrapeptide. Analogs of the tetrapeptide rigin useful in accordance with the present invention include those in which one or more of the four amino acids are reorganized or rearranged within the sequence and/or where no more than two of the amino acids are substituted (e.g., Ala-Gln-Thr-Arg(SEQ ID NO:2). More preferably, at least one of the amino acids within the sequence is Pro or Arg and most preferably the tetrapeptide includes both Pro and Arg although their order and position may vary. The amino acid substitutions can be from amongst any amino acid as defined herein. Particularly preferred rigin-based tetrapeptides include Xaa-Xbb-Arg-Xcc, Xaa-Xbb-Xcc-Pro, Xaa-Xbb-Pro-Arg, wherein Xaa-Xbb-Pro-Xcc, Xaa-Xbb-Xcc-Arg, Xaa, Xbb and Xcc may be the same or different and selected from the following Xaa is Gly or the amino acids that may be substituted therefore, Xbb is Gln or the amino acids that may be substituted therefore and Xcc may be Pro or Arg or the amino acids substituted therefore. The most preferable amino acids substituted for Gly include an aliphatic side chain such as, without limitation, beta-Ala, Ala, Val, Leu, Pro, Sarcosine (Sar) and Ile. The most preferable amino acids substituted for Gln include a side chain that includes an amine group that is predominantly uncharged at neutral pH (pH 6-7) such as, without limitation, Asn, Lys, Orn, 5-hydroxyproline, Citrulline and Canavanine. When Arg is substituted, it is preferably replaced with an amino acid having a side chain that includes, predominantly, a charged nitrogen at a pH of 6, such as, without limitation, Pro, Lys, His, Desmosine and Isodesmosine.

Derivatives are also considered to be encompassed by the term rigin-base tetrapeptides in accordance with the present invention, (and therefore also the more generic term tetrapeptides). Derivatives include derivatives of the substituted and rearranged rigin-based tetrapeptides described herein. These derivatives include, inter alia, acyl-derivatives, which are tetrapeptides substituted with one or more straight-chain or branched-chain, long or short chain, saturated or unsaturated, substituted with a hydroxy, amino, amino acyl, sulfate or sulfide group or unsubstituted having from 1 to 29 carbon atoms. N-acyl-derivatives include those acyl groups which can be derived from acetic acid, capric acid, lauric acid, myristic acid, octanoic acid, palmitic acid, stearic acid, behenic acid, linoleic acid, linolenic acid, lipoic acid, oleic acid, isostearic acid, elaidoic acid, 2-ethylhexaneic acid, coconut oil fatty acid, tallow fatty acid, hardened tallow fatty acid, palm kernel oil fatty acid, lanolin fatty acid and the like. Preferable examples of the acyl group include an acetyl group, a palmitoyl group, an elaidoyl group, a myristyl group, a biotinyl group and an octanoyl group. These may be substituted or unsubstituted. When substituted, they are preferably substituted with hydroxyl or sulphur comprising groups such as, without limitation SO3H, SH or S—S.

ALAMCAT tetrapeptides are tetrapeptides which include at least one amino acid including an aliphatic group comprising side chain. These amino acids include, without limitation, Gly, beta-Ala, Ala, Val, Leu, Sarcosine (Sar) and Ile. These tetrapeptides also include at least one amino acid including at least one NH2 comprising side chain. These amino acids include a side chain that has an amine group that is predominantly uncharged at neutral pH (pH 6-7) such as, without limitation, Gln, Asn, Lys, Om, 5-hydroxyproline, Citrulline and Canavanine. The ALAMCAT-tetrapeptides also include at least one amino acid having at least one side chain including at least one cationic amine (predominant species is charged such as NH3+, NH2+, etc.—basic amino acids which are positively charged at pH 6.0). These amino acids include, without limitation, Pro, Arg, Lys, His, Desmosine and Isodesmosine. The remaining amino acid can be any amino acid, but is preferably one comprising an alphatic group, pendant amino group or pendant cationic group. Derivatives are also considered to be encompassed by the term ALAMCAT-tetrapeptides in accordance with the present invention, (and therefore also the more generic term tetrapeptides). These derivatives include, inter alia, acyl-derivatives, which are tetrapeptides substituted with one or more straight-chain or branched-chain, substituted or unsubstituted long or short chain, saturated or unsaturated acyl group(s) having from 1 to 29 carbon atoms. The acyl groups which can be used are the same as those described for the rigin-based tetrapeptides.

Preferred embodiments include Peptide E, arg-ser-arg-lys (SEQ ID NO:3), N-acyl-Gly-Gln-Pro-Arg (SEQ ID NO:1) peptides, most preferably N-palmitoyl-Gly-Gln-Pro-Arg(SEQ ID NO:1).

Preferred commercially available sources of tetrapeptides include RIGIN, EYELISS, Haloxyl, and MATRIXYL 3000, which comprise between 50 to 500 ppm of palmitoyl-Gly-Gln-Pro-Arg(SEQ ID NO:1), and other ingredients, such as peptides, chalcones and an excipient, commercially available from SEDERMA, France. Tego Pep 417 available from Evonik. These may be used to produce compositions of the present invention by adding thereto at least one tripeptide as described herein.

The tetrapeptide where present (e.g. a lipo-peptide) may have an amino acid sequence selected from the group consisting of: (i) GPXG, (ii) LSXX (iii) XXGD or (iv) QTAV (SEQ ID NO:4)

wherein for (i) G denotes the amino acid glycine and P denotes the amino acid proline (as per the internationally recognised single letter code for amino acids), and X denotes an amino acid selected from the group consisting of Lysine (K), Glutamic acid (E) and Serine (S) and mixtures thereof, wherein for (ii) L denotes the amino acid Leucine and S denotes the amino acid Serine (as per the internationally recognised single letter code for amino acids), and X denotes an amino acid selected from the group consisting of Valine (V), Aspartic acid (D), Proline (P), Glycine (G) and mixtures thereof, wherein for (iii) G denotes the amino acid Glycine and D denotes the amino acid Aspartic acid (as per the internationally recognised single letter code for amino acids), and X denotes an amino acid selected from the group consisting of Glutamic acid (E), Lysine (K), Leucine (L), Alanine (A), Isoleucine (I), Arginine (R) and mixtures thereof, and for (iv) Q denotes the amino acid Glutamine, T denotes the amino acid Threonine, A denotes the amino acid Alanine, and V denotes the amino acid Valine.

In another embodiment, the peptide (e.g. a lipo-peptide) has an amino acid sequence selected from the group consisting of: (i) Y-GPXG-Z, (ii) Y-LSXX-Z (iii) Y-XXGD-Z or (iv) Y-QTAV-Z (SEQ ID NO:4) wherein for (i) G denotes the amino acid glycine and P denotes the amino acid proline (as per the internationally recognised single letter code for amino acids), and X denotes an amino acid selected from the group consisting of Lysine (K), Glutamic acid (E) and Serine (S) and mixtures thereof, wherein for (ii) L denotes the amino acid Leucine and S denotes the amino acid Serine (as per the internationally recognised single letter code for amino acids), and X denotes an amino acid selected from the group consisting of Valine (V), Aspartic acid (D), Proline (P), Glycine (G) and mixtures thereof, wherein for (iii) G denotes the amino acid Glycine and D denotes the amino acid Aspartic acid (as per the internationally recognised single letter code for amino acids), and X denotes an amino acid selected from the group consisting of Glutamic acid (E), Lysine (K), Leucine (L), Alanine (A), Isoleucine (I), Arginine (R) and mixtures thereof, wherein for (iv) Q denotes the amino acid Glutamine, T denotes the amino acid threonine, A denotes the amino acid Alanine, and V denotes the amino acid Valine, and wherein at the N-terminal end, Y is selected from the group consisting of H, —CO—R¹, —SO₂—R¹ or a biotinyl group, at the C-terminal end, Z is selected from the group consisting of OH, O R¹, NHR¹ or NR¹R².

In one embodiment, R¹ and R² are independently selected from the group consisting of alkyl, aryl, aralkyl, alkylaryl, alkoxy, saccharide and aryloxy group, which may be linear, branched, cyclical, polycyclic, unsaturated, hydroxylates, carbonylated, phosphorylated and/or sulphurous, said groups comprising from 1 to 24 carbon atoms and being capable of including one or more heteroatoms 0, S and/or N.

In a preferred embodiment of the present invention the tetrapeptide is modified at the N-terminal and/or the C-terminal end.

In a preferred embodiment the tetrapeptides of the present invention is selected from the group consisting of: Y-EKGD-Z (SEQ ID NO:5), Y-ELGD-Z (SEQ ID NO:6), Y-EAGD-Z (SEQ ID NO:7), Y-EIGD-Z (SEQ ID NO:8), Y-ERGD-Z (SEQ ID NO:9), Y-KEGD-Z (SEQ ID NO:10), Y-KLGD-Z (SEQ ID NO:11), Y-KAGD-Z (SEQ ID NO:12), Y-KIGD-Z (SEQ ID NO:13), Y-KRGD-Z (SEQ ID NO:14), Y-LEGD-Z (SEQ ID NO:15), Y-LKGD-Z (SEQ ID NO:16), Y-LAGD-Z (SEQ ID NO:17), Y-LIGD-Z (SEQ ID NO:18), Y-LRGD-Z (SEQ ID NO:19), Y-IEGD-Z (SEQ ID NO:20), Y-IKGD-Z (SEQ ID NO:21), Y-ILGD-Z (SEQ ID NO:22), Y-IAGD-Z (SEQ ID NO:23), Y-IRGD-Z (SEQ ID NO:24), Y-REGD-Z (SEQ ID NO:25), Y-RKGD-Z (SEQ ID NO:26), Y-RLGD-Z (SEQ ID NO:27), Y-RAGD-Z (SEQ ID NO:28), Y-RIGD-Z (SEQ ID NO:29), Y-AEGD-Z (SEQ ID NO:30), Y-AKGD-Z (SEQ ID NO:31), Y-ALGD-Z (SEQ ID NO:32), Y-AIGD-Z (SEQ ID NO:33) and Y-ARGD-Z (SEQ ID NO:34). In another embodiment, the tetrapeptides is selected from the group consisting of Y-EKGD-Z (SEQ ID NO:5), Y-LKGD-Z (SEQ ID NO:16), Y-IRGD-Z (SEQ ID NO:24) and Y-AKGD-Z (SEQ ID NO:31). In another embodiment the tetrapeptide is Y-EKGD-Z (SEQ ID NO:5). In another embodiment the tetrapeptide is Y-LKGD-Z (SEQ ID NO:16). In another embodiment the tetrapeptide is Y-IRGD-Z (SEQ ID NO:24). In another embodiment the tetrapeptide is Y-AKGD-Z (SEQ ID NO:31).

In a preferred embodiment the tetrapeptide combination of the present invention, tetrapeptide a) is selected from the group consisting of: Y-LSVD-Z (SEQ ID NO:35), Y-LSVP-Z (SEQ ID NO:36), Y-LSVG-Z (SEQ ID NO:37), Y-LSDV-Z (SEQ ID NO:38), Y-LSDP-Z (SEQ ID NO:39), Y-LSDG-Z (SEQ ID NO:40), Y-LSPV-Z (SEQ ID NO:41), Y-LSPD-Z (SEQ ID NO:42), Y-LSPG-Z (SEQ ID NO:43), Y-LSGV-Z (SEQ ID NO:44), Y-LSGD-Z (SEQ ID NO:45) and Y-LSGP-Z (SEQ ID NO:46). In another embodiment, the tetrapeptides is selected from the group consisting of Y-LSVD-Z (SEQ ID NO:35), Y-LSPG-Z (SEQ ID NO:43) and Y-LSPD-Z (SEQ ID NO:42). In another embodiment the tetrapeptide is Pal-LSVD-OH(SEQ ID NO:35). In another embodiment the tetrapeptide is Pal-LSPG-OH(SEQ ID NO:43). In another embodiment the tetrapeptide is Pal-LSPD-OH(SEQ ID NO:42).

In a further preferred embodiment of the present invention, the tetrapeptide is Y-GPKG-Z (SEQ ID NO:47). In a further preferred embodiment of the present invention the tetrapeptide is Y-GPEG-Z (SEQ ID NO:48). In a further preferred embodiment of the present invention the tetrapeptide is Y-GPSG-Z (SEQ ID NO:49).

The tetrapeptides of the present invention are preferably used in amounts from 0.1 ppm (0.00001% w/w also referred to herein as “weight percent”, “weight %” or simply by weight) to 10,000 ppm (0.5% w/w), preferably from 0.5 ppm to 1000 ppm (0.05% w/w), and most preferably from 1 ppm to 500 ppm by weight of the composition.

The combination of tripeptides and tetrapeptides, is particularly preferred. The preferred ratio of tetrapeptide to tripeptide, or indeed the ratio of molecules having four amino acids to those having three amino acids can range from 100:1 to 1:100; more preferably from 50:1 to 1:50, even more preferably from 30:1 to 1:30 and even more preferably between 10:1 to 1:10. Most preferably, the ratio of tetrapeptide to tripeptide ranges from between 3:1 to 1:3. These ratios are on a weight basis (% w/w—e.g. mg of pure peptide per Kilogram in the final formulation). In a particularly preferred embodiment, the amount of tripeptide used is greater than the amount of tetrapeptide used when considered in terms of their amounts in parts per million, again based on overall weight of the composition. In a particularly preferred embodiment, the emulsion of the present invention comprise a tetrapeptide of the sequence Gly-Gln-Pro-Arg(SEQ ID NO:1), its analogs and derivatives in combination with one or more tripeptide of the sequences Gly-His-Lys, its analogs and derivatives.

### Pentapeptides

The compositions of the present invention may optionally comprise a pentapeptide, derivatives of pentapeptides, and mixtures thereof. As used herein, “pentapeptides” refers to both the naturally occurring pentapeptides and synthesized pentapeptides. Also useful herein are naturally occurring and commercially available compositions that comprise pentapeptides. Suitable pentapeptides are those selected from the group consisting of pentapeptide 1, 4, 5, 6, 7, 8, 9, 10, 11, 12, 13, 14, 15, 16, 17, 18, 19, 21, 22, 23, 24, 25, 26, 28, 29, 30, 31, 33, 34, 35, 36, 38, 39, derivatives thereof and mixtures thereof.

Suitable pentapeptides for use herein are the pentapeptide, lys-thr-thr-lys-ser (SEQ ID NO:50), Arg-asp-lys-tyr-val (SEQ ID NO:51) (pentapeptide-1) and derivatives thereof. A preferred commercially available pentapeptide derivative-comprising composition is Matrixyl which comprises 100 ppm of palmitoyl-lys-thr-thr-lys-ser (SEQ ID NO:50) and is commercially available from Sederma, France.

The pentapeptides and/or pentapeptide derivatives where present are preferably included in emulsion at amounts of from 0.01% to 20%, more preferably from 0.05% to 15%, and even more preferably from 0.1% to 10%, by weight of the emulsion composition.

### Matrix Metalloproteinase Inhibitors (MMPi)

The composition of the present invention may comprise a matrix metalloproteinase inhibitor. The term “matrix metalloproteinase inhibitor” relates to all molecule and/or plant or bacterial extracts having an inhibitory activity on at least one of the matrix metalloproteinases expressed or synthetized by or in the skin. The family of the matrix metalloproteinases is formed of several well-defined groups on the basis of their resemblance regarding structure and substrate specificity (Woessner J. F., Faseb Journal, vol. 5, 1991, 2145). Among these groups, there are collagenases able to degrade fibrillar collagens (MMP-1 or interstitial collagenase, MMP-8 or neutrophil collagenase, MMP-13 or collagenase 3, MMP-18 or collagenase 4), gelatinases degrading type IV collagen or other denatured collagen form (MMP-2 or A gelatinase (72 kDa), MMP-9 or B gelatinase (92 kDa)), stromelysins (MMP-3 or stromelysin 1, MMP-10 or stromelysin 2, MMP-11 or stromelysin 3) whose broad spectrum of activity targets proteins of the extracellular matrix such as glycoproteins (fibronectin, laminin), proteoglycanes etc., matrilysin (MMP-7), metalloelastase (MMP-12) or metalloproteinases (MMP-14, MMP-15, MMP-16 and MMP-17). Metalloproteinases (MMPs) are proteases that use a metal, (mostly zinc) coordinated to 3 cystein residues and to a methionine in their active site, that degrade macromolecular components of the extracellular matrix and of basal layers at neutral pH (collagen, elastin, etc. . . . ). This group of enzymes is inactivated by metal chelators. The principal activity regulators of MMPs are the tissue inhibitors of metalloproteinases or TIMPs such TIMP-I, TIMP-2, TIMP-3 and TIMP-4 (Woessner J. F., Faseb Journal, 1991). Furthermore, the MMPs expression is also regulated by growth factors, cytokins, oncogens products (ras, jun), or also matrix constituents.

The term “matrix metalloproteinase inhibitors” according to the present invention means all molecules able to reduce the MMPs activity regarding the gene expression (transcription and translation) or regarding the activation of the zymogen form of MMPs, or else regarding the local control of active forms. Furthermore, the metalloproteinase inhibitors according to the present invention can also be MMP-1 inhibitors of natural or synthetic origin. The terms “natural origin” or “synthetic origin” mean both a metalloproteinase inhibitor at a pure state or in solution at different concentrations, but natural origin termed inhibitors are obtained by different extraction methods from a natural element (for example lycopene from a tomato) whereas the inhibitors of synthetic origin are all obtained via chemical synthesis

Preferred MMPi are selected from the group consisting of retinoid, N-acetyl cysteine, glutathione, 2-furildioxime, vitamin C, flavones, isoflavones, hydrolysed rice protein, alfalfa extract, white lupin, Zizyphus jujube extract, dihydroxy methyl chromone, kudzu extract, Vitis vinifera extract, Oenothera biennis extract Anogeissus leiocarpus extract and mixtures thereof.

Where present MMPi are present at a level of from 0.01% to 10%, more preferably 0.1% to 5% and most preferably from 1% to 2.5% by weight of the composition.

### Skin Conditioning Agent

The compositions of the present invention may optionally comprise a skin conditioning agent. Said skin conditioning agents may preferably be selected from the group consisting of humectants, emollients, moisturisers, or mixtures thereof. Where present, they are preferably present at a level of from 0.01% to 20%, more preferably from 0.1% to 10%, most preferably from 0.5% to 7% by weight of the emulsion.

Preferred skin conditioning agents are selected from the group consisting of guanidine, urea, glycolic acid and glycolate salts, salicylic acid, lactic acid and lactate salts, aloe vera, shea butter, polyhydroxy alcohols, such as sorbitol, mannitol, xylitol, erythritol, glycerol, hexanetriol, butanitriol, (di) propylene glycol, butylene glycol, hexylene glycol, polyethylene glycol, sugars (e.g. fructose, glucose, xylose, honey, mannose, xylose), gluconodeltalactone, and starches and their derivatives, pyrrolidone, carboxylic acid, hyaluronic acid and salts thereof, lactamide monoethanolamine, acetamide monoethanolamine, panthenol, allantoin and mixtures thereof.

The composition may comprise hyaluronic acid or a salt thereof, such as sodium hyaluronate or potassium hyaluronate. The composition preferably comprises sodium hyaluronate.

In particular, the hyaluronic acid or salt thereof (e.g. sodium hyaluronate) may be present in the composition in an amount by weight of from 0.01% to 0.2% or from 0.03% to 0.1%.

More preferably said skin conditioning agent is selected from glycerine, arabinogalactan, butylene glycol, hyaluronic acid, shea butter, propylene glycol, ethylhexyl glycerin and hyaluronate.

### Vitamins

The compositions of the present invention may comprise one or more vitamins. The compositions may comprise ascorbates, for example vitamin C, vitamin C derivatives, ascorbic acid, ascorbyl glucoside, ascorbyl palmitate, magnesium ascorbyl phosphate, sodium ascorbyl phosphate and ethyl ascorbic acid. The composition may comprise vitamin B, vitamin B derivatives, vitamin B1 to vitamin B12 and their derivatives. In a further embodiment the composition comprising the Vitamin B3 derivative niacinamide.

In an alternative embodiment of the present the cosmetic composition comprises vitamin K, vitamin K derivatives, vitamin H, vitamin D, vitamin D derivatives and mixtures thereof. In an alternative embodiment of the present the cosmetic composition comprises vitamin E, vitamin E derivatives such as tocopherol and tocopheryl acetate, and provitamins thereof, such as panthenol and mixtures thereof.

In a further embodiment the present cosmetic composition comprises retinoid compounds, including retinoic acid, retinaldehyde, retinol and derivatives thereof. In one embodiment the cosmetic composition comprises retinyl palmitate, retinyl acetate, retinyl retinoate, retinyl proprionate, retinyl ascorbate, retinyl linoleate, retinyl retinoate, retinyl sunflowerseedate and mixtures thereof.

The vitamin compounds may be included as the substantially pure material, or as an extract obtained by suitable physical and/or chemical isolation from natural (e. g. plant) sources. In one embodiment, when vitamin compounds are present in the compositions of the instant invention, the emulsion compositions comprise from about 0.0001% to 50%, more preferably from 0.001% to 10%, still more preferably from 0.01% to 8%, and still more preferably from 0.1% to 5%, by weight of the composition, of the vitamin compound.

### Sunscreen

The compositions of the present invention may optionally comprise a sunscreen component. The sunscreen may comprise organic or inorganic sun filters or a combination of the two. Suitable inorganic sunfilters include those selected from the group consisting of microfine titanium dioxide, microfine zinc oxide, boron nitride and mixtures thereof.

Suitable organic sunscreens include those selected from the group consisting of: a) p-aminobenzoic acids, their esters and derivatives (for example, 2ethylhexyl p-dimethylaminobenzoate), b) methoxycinnamate esters (for example, 2-ethylhexyl p-methoxycinnamate, 2-ethoxyethyl p-methoxycinnamate or a, p-di-(p-methoxycinnamoyl)-a′-(2ethylhexanoyl)-glycerin, c) benzophenones (for example oxybenzone), d) dibenzoylmethanes such as 4-(tert-butyl)-4′-methoxydibenzoylmethane, e) 2-phenylbenzimidazole-5 sulfonic acid and its salts, f) alkyl-ss, ss-diphenylacrylates for example alkyl a-cyano-ss, ss-diphenylacrylates such as octocrylene, g) triazines such as 2,4,6-trianilino-(p-carbo-2-ethyl-hexyl-1-oxi)-1, 3,5 triazine, h) camphor derivatives such as methylbenzylidene camphor and i) mixtures thereof. Other preferred sunscreen ingredients include those selected from the group consisting of homosalate, Ethylhexyl salicylate, Diethylhexylbutamido triazone, Bis-ethylhexyloxyphenol methoxyphenyl triazine, Diethylamino hydroxybenzoyl hexyl benzoate, Butyl methoxydibenzoylmethane, Methylene bis-benzotriazoyl tetramethylbutylphenol, Polysilicone-15 and mixtures thereof. A sunscreening agent is optionally present in an amount from 0.1 to 10% by weight of the composition.

### Other Optional Ingredients

The compositions of the present invention may also optionally comprise one or more of the following optional ingredients. Preservatives may be added to the emulsion such as 2-bromo2-nitropropane-1,3-diol (bronopol, which is available commercially under the trade name Myacide®), benzyl alcohol, diazolidinyl urea, imidazolidinyl urea, methyl paraben, phenoxy ethanol, ethyl paraben, propyl paraben, sodium methyl paraben, sodium dehydroacetate, polyhexamethylenebiguanide hydrochloride, isothiazolone and sodium propyl paraben, suitably in an amount of from 0.01% to 10% by weight of the emulsion.

Thickeners, viscosity modifying agents and/or gelling agents may be added to the emulsion composition, such as acrylic acid polymers e. g. available commercially under the trade name Carbopol, Pemulen or Ultrez (Lubrizol) or modified celluloses e. g. hydroxyethylcellulose available commercially under the trade name Natrosol (Hercules) or hydroxypropylmethyl cellulose, amine oxides, block polymers of ethylene oxide and propylene oxide (for example, those available from BASF Wyandotte under the trade name Pluronic”®), PVM, MA, or a decadiene crosspolymer (available under the trade name Stabilez 60), ethoxylated fatty alcohols, salt (magnesium chloride, sodium chloride), Aristoflex AVC (Clariant), phthalic acid amide, xanthan gum, sodium polyacrylate, polyvinyl alcohols, fatty alcohols and alkyl galactomannans available under the trade name N-Hance from Hercules, suitably in an amount of from 0.5% to 10% by weight of the composition.

Sequestering agents may be added to the emulsion composition, such as ethylenediamine tetraacetic acid and salts thereof, suitably in an amount of from 0.005% to 0.5% by weight of the composition.

The composition may also include waxes such as cocoa butter suitably in an amount of from 1% to 99% by weight of the composition.

The composition may also comprise suitable, cosmetically acceptable diluents, carriers and/or propellants such as dimethyl ether.

The composition may also include pearlising agents such as stearic monoethanolamide and/or mica, suitably in an amount of from 0.01% to 10% by weight of the composition.

Perfumes may be added suitably in an amount of from 0.01% to 2% by weight of the composition, as may water soluble dyes such as tartrazine, suitably in an amount of from a trace amount (such as 1×10-5%) to 0.1% by weight of the composition.

The composition may also include pH adjusting agents such as sodium hydroxide, aminomethyl propanol, triethanolamine, suitably in an amount of from 0.01% to 10% by weight of the composition. The composition may be buffered by means well known in the art, for example by use of buffer systems comprising succinic acid, citric acid, lactic acid, and acceptable salts thereof, phosphoric acid, mono- or disodium phosphate and sodium carbonate. Suitably, the composition may have a pH between 3 and 10, preferably between 4 and 8.

### Methods

Peri- and post-menopausal skin demonstrates a decrease in the quality of the skin barrier function partly due to an impaired differentiation process that is critical to the transformation of keratinocytes into corneocytes. Transglutaminase, loricrin and involucrin are examples of measurable biomarkers of keratinocyte differentiation. The effectiveness of the presently described compositions in improving the differentiation process and thereby promoting a healthy skin barrier are assessed by measuring the increase in the expression of differentiation biomarkers in the skin cells tested. An under expression of these markers in the epidermis can lead to disruption in epidermal homeostasis by reducing skin cell differentiation,

In the present experiment, the effects of compounds were assessed in normal human epidermal keratinocytes (NHEK). More specifically, the effects of these compounds were evaluated using real time polymerase chain reaction (RT-qPCR) technology. A PCR array designed by Bioalternatives for the analysis of 32 target genes (including 2 housekeeping genes) was used to determine the gene expression pattern in the NHEK cells through the use of the ribonucleic acid (mRNA) extracted from the treated and control cells.

Normal human epidermal keratinocytes (NHEK) were seeded in 24-well plates and placed in culture medium (Keratinocyte-serum free media (SFM) supplemented with Epidermal Growth Factor at concentration 0.025 ug/ml and Pituitary extract at concentration of 0.05 mg/ml) for 24 hours. To determine suitable non-toxic ingredient concentrations, a cytotoxicity assay was first carried out where the NHEKs were treated with ingredients or ingredient blends for 24 hours before being incubated with WST-8 (highly water-soluble tetrazolium salt) which is reduced to a water-soluble orange coloured product (formazan) by viable, living cells and is quantified by optical density measurement at 450 nm using a spectrophotometer (VERSAmax, Molecular Devices). This reduction is proportional to the number of living cells and their metabolic activity

For the RT-qPCR study, after incubating the NHEKs in culture medium for 24 hours, the medium was then removed and replaced by assay medium (Keratinocyte-SFM) alone, as a negative control, or containing the test compounds and the cells were then incubated for 24 hours. Experiments were performed in triplicate. After incubation, cells were washed in phosphate buffered saline (PBS) solution and immediately frozen at −80° C.

Epidermal differentiation markers mRNA expression was analysed using RT-qPCR. Total mRNA was extracted from each sample using TriPure Isolation Reagent (trade name) according to the supplier's instructions (Sigma-Aldrich). The amount and quality of mRNA was evaluated using capillary electrophoresis with a Bioanalyzer 2100 (Agilent technologies). Before reverse transcription, the mRNA was purified using Chemagic™ mRNA Direct kit commercially available from Perkin Elmer. The complementary DNA (cDNA) was synthetized by reverse transcription of total mRNA or mRNA in presence of oligo(dT) and Transcriptor Reverse Transcriptase (tradename, commercially available from Roche). The cDNA quantities were then diluted if necessary, to achieve equal amount of cDNA concentration in each test sample, before carrying out PCR (Polymerase Chain Reaction) using the LightCycler (trade name) system (commercially available from Roche Molecular System Inc.) according to the supplier's instructions. The reaction mix (10 μl final) was prepared as follows: —2.5 μl of cDNA, —primers (forward and reverse), —reagent mix containing taq DNA polymerase, SYBR Green I (Asymmetrical cyanine dye—Thermo Fisher Scientific) and MgCl2. SYBR Green is a DNA fluorescent dye which will bind in a specific groove of the DNA molecule. When a laser is fired at it, it will glow providing a measurable marker. The more copies of DNA that have been produced through PCR, the more SYBR Green molecules will bind the DNA and the higher the level of fluorescence. The incorporation of fluorescence in amplified DNA was continuously measured during the PCR cycles to provide a “fluorescence intensity allowing the evaluation of a relative expression (RE) value for the mRNA of the different differentiation markers. Only the differentiation marker cDNA is amplified due to the specific primers used and hence the number of copies of cDNA produced following each round of amplification of the PCR is proportional to the amount of cDNA/mRNA that was originally present in the cells. The incorporation of fluorescence in the amplified cDNA was continuously measured during the PCR cycles to provide a “fluorescence intensity” allowing the evaluation of a relative expression (RE) value for the differentiation marker mRNA of the test compound treated cells compared with the media only treated/negative control cells.

### REPRESENTATIVE COMPOSITION EXAMPLES

The composition examples of the present invention are not meant to be limiting.

### Example 1—Water in Oil Emulsion

| Material | % w/w |
|---|---|
| Dimethicone | 13.83 |
| Water | 35.11 |
| Glycerin | 5.00 |
| Dimethicone crosspolymer & | 31.31 |
| Dimethcone | |
| Butylene glycol | 2.60 |
| PEG/PPG-18/18 dimethicone & | 3.00 |
| Polyglyceryl-4 isostearate & Hexyl | |
| laurate | |
| Cetyl PEG/PPG-10/1 dimethicone | 2.00 |
| Magnesium sulphate | 0.60 |
| Phenoxyethanol & Caprylyl | 0.55 |
| glycol & Ethylhexylglycerin | |
| Ceramide complex | 1.00 |
| (blend of Cholesterol & | |
| Ceramide AP & | |
| Ceramide EOP & Ceramide | |
| NP & Phytosphingosine) | |
| Niacinamide | 1.00 |
| Ophiopogon japonicus | 3.00 |
| root extract & Water | |
| Soy isoflavones & | 1.00 |
| Genistein & Water & | |
| Lecithin | |

### Method of Manufacture

1. In the main vessel add Dimethicone, Dimethicone crosspolymer, PEG/PPG-18/18 dimethicone & polyglyceryl-4 isostearate & hexyl laurate and Cetyl PEG/PPG-10/1 dimethicone to make the oil phase.
2. Weigh out water, magnesium sulphate, glycerin, phenoxyethanol & caprylyl glycol & ethylhexylglycerin, Niacinamide, Ophiopogon japonicus root extract & Water, Ceramide complex and Soy isoflavones & Genistein & Water & Lecithin stir until solids are dissolved to make the water phase.
3. Add the water phase to the oil phase slowly with constant stirring at high speed (creating a vortex). Continue stirring for 5 minutes.
4. Homogenise the product for 5 minutes at 3500 rpm using a Silverson mixer or equivalent.

### Example 2—Oil in Water Emulsion

| Material | % w/w |
|---|---|
| Dimethicone | 3.50 |
| Octyldodecanol | 2.00 |
| Water | 75.95 |
| Glycerin | 5.00 |
| Aluminium starch octentylsuccinate | 1.00 |
| Dimethicone crosspolymer & | 1.00 |
| Dimethicone | |
| Phenoxyethanol & Caprylyl | 0.80 |
| glycol & Ethylhexylglycerin | |
| Glyceryl stearate & | 2.00 |
| PEG-100 stearate | |
| Cetearyl alcohol | 2.00 |
| Ammonium acryloyl- | 0.60 |
| dimethyltaurate/VP | |
| copolymer | |
| Xanthan gum | 0.10 |
| Tetrasodium EDTA | 0.05 |
| Ceramide complex (blend of | 1.00 |
| Cholesterol & Ceramide AP & | |
| Ceramide EOP & Ceramide | |
| NP & Phytosphingosine) | |
| Niacinamide | 1.00 |
| Ophiopogon japonicus root | 3.00 |
| extract & Water | |
| Soy isoflavones & Genistein & | 1.00 |
| Water & Lecithin | |

### Method of Manufacture

1. To water add glycerin and dissolve tetrasodium EDTA.
2. Using homogenisation sprinkle in xanthan gum and continue to homogenise for 5 minutes or until hydrated.
3. Heat water phase to 70-75° C.
4. In a separate vessel weigh out oil phase and heat to 70-75° C. (Dimethicone, cetearyl alcohol, Octyldodecanol, glyceryl stearate & PEG-100 stearate) When at temperature stir in the Ammonium acryloyldimethyltaurate/VP copolymer and Aluminium starch octentylsuccinate.
5. With both phases at 70-75° C. add the oil phase to the water phase and homogenise for 2 minutes.
6. Add dimethicone crosspolymer & dimethicone and homogenise for 2 minutes.
7. Cool to room temperature.
8. Stir in Phenoxyethanol & Caprylyl glycol & Ethylhexylglycerin, Ophiopogon japonicus root extract & Water, Ceramide complex and Soy isoflavones & Genistein & Water & Lecithin.
9. Dissolve Niacinamide in a small amount of water and stir into the bulk.
10. Make to weight with water and stir smooth.

### Example 3—Oil in Water Emulsion Containing Sunscreens

| Material | % w/w |
|---|---|
| C12-15 alkyl benzoate | 5.00 |
| Butyl methoxydibenzoylmethane | 3.00 |
| Ethylhexyl methoxycinnamate | 5.00 |
| Cetearyl alcohol | 2.00 |
| Glycerin | 2.00 |
| Glyceryl stearate & | 3.00 |
| PEG-100 stearate | |
| Dimethicone | 1.50 |
| PEG-20 stearate | 0.50 |
| Phenoxyethanol & Caprylyl | 0.40 |
| glycol & Ethylhexylglycerin | |
| Carbomer | 0.20 |
| Potassium hydroxide | 0.06 |
| Tetrasodium EDTA | 0.05 |
| Water | 71.29 |
| Ceramide complex | 1.00 |
| (blend of Cholesterol & | |
| Ceramide AP & | |
| Ceramide EOP & Ceramide | |
| NP & Phytosphingosine) | |
| Niacinamide | 1.00 |
| Ophiopogon japonicus | 3.00 |
| root extract & Water | |
| Soy isoflavones & | 1.00 |
| Genistein & Water & Lecithin | |

### Method of Manufacture

1. To water add glycerine and dissolve tetrasodium EDTA.
2. Using homogenisation sprinkle in carbomer and continue to homogenise for 5 minutes or until hydrated.
3. Heat water phase to 70-75° C.
4. In a separate vessel weigh out oil phase and heat to 70-75° C. (Dimethicone, cetearyl alcohol, glyceryl stearate & PEG-100 stearate, C12-15 alkyl benzoate, Butyl methoxydibenzoylmethane, Ethylhexyl methoxycinnamate, PEG-20 stearate)
5. With both phases at 70-75° C. add the oil phase to the water phase and homogenise for 2 minutes.
6. Add Potassium hydroxide and homogenise for 2 minutes.
7. Cool to room temperature.
8. Stir in Phenoxyethanol & Caprylyl glycol & Ethylhexylglycerin and Ophiopogon japonicus root extract, Ceramide complex & Water and Soy isoflavones & Genistein & Water & Lecithin.
9. Dissolve Niacinamide in a small amount of water and stir into the bulk.
10. Make to weight with water and stir smooth.

### Example 4—Gel

| Material | % w/w |
|---|---|
| Glycerin | 5.00 |
| Propanediol | 2.00 |
| Acrylates/C10-30 alkyl | 1.00 |
| acrylate crosspolymer | |
| Phenoxyethanol & | 0.40 |
| Caprylyl glycol & | |
| Ethylhexylglycerin | |
| Potassium hydroxide | 0.29 |
| Tetrasodium EDTA | 0.05 |
| Water | 85.26 |
| Ceramide complex | 1.00 |
| (blend of Cholesterol & | |
| Ceramide AP & | |
| Ceramide EOP & Ceramide | |
| NP & Phytosphingosine) | |
| Niacinamide | 1.00 |
| Ophiopogon japonicus | 3.00 |
| root extract & Water | |
| Soy isoflavones & | 1.00 |
| Genistein & Water & Lecithin | |

### Method of Manufacture

1. To water add glycerine and propanediol and dissolve tetrasodium EDTA.
2. Using homogenisation sprinkle in acrylates/C10-30 alkyl acrylate crosspolymer and continue to homogenise for 5 minutes or until hydrated.
3. Stir in potassium hydroxide to form gel.
4. Stir in Phenoxyethanol & Caprylyl glycol & Ethylhexylglycerin, Ophiopogon japonicus root extract & Water, Ceramide complex and Ophiopogon japonicus root extract & Water.
5. Dissolve Niacinamide in a small amount of water and stir into the bulk.
6. Make to weight with water and stir smooth.

### Example 5—Detergent Wash

| Material | % w/w |
|---|---|
| Sodium laureth sulphate | 36 |
| Cocamidopropyl betaine | 4 |
| Cocamide DEA | 1.5 |
| Sodium chloride | 1.5 |
| Sodium benzoate | 0.3 |
| Tetrasodium EDTA | 0.05 |
| Citric acid | 0.05 |
| Sodium hydroxide | 0.05 |
| Water | 49.15 |
| Ceramide complex | 1.00 |
| (blend of Cholesterol & | |
| Ceramide AP & | |
| Ceramide EOP & | |
| Ceramide | |
| NP & Phytosphingosine) | |
| Niacinamide | 1.00 |
| Ophiopogon japonicus | 3.00 |
| root extract & Water | |
| Soy isoflavones & | 1.00 |
| Genistein & Water & Lecithin | |

### Method of Manufacture

1. To water tetrasodium EDTA and sodium chloride.
2. Stir in sodium laureth sulfate, cocamidopropyl betaine, cocamide DEA.
3. Dissolve sodium benzoate in a small amount of water and stir into bulk,
4. Stir in Ophiopogon japonicus root extract & Water, Ceramide complex and Soy isoflavones & Genistein & Water & Lecithin.
5. Dissolve Niacinamide in a small amount of water and stir into the bulk.
6. Add sodium hydroxide and citric acid to achieve desired pH.
7. Make to weight with water and stir smooth

Additional details are provided below for some commercially available materials that are sold under the identified trade marks.

TABLE
of commercially available materials

| | | Active | % active | Active | |
|---|---|---|---|---|---|
| Supplier | Trade name | INCI | INCI | compound | Excipients |
| Mibelle | Lipobelle ™ | Soy | | Genistein | Alcohol, |
| | Soyaglycone | Isoflavones | | | lecithin, |
| | | | | | Polsorbate |
| | | | | | 80 & Aqua |
| DSM | Regu ®-Fade | Resveratrol | 100% | Trans | None |
| | | | | resveratrol | |
| Lipotec | Lipochroman ™ | Dimethyl- | 100% | Dimethyl- | None |
| | | methoxy | | methoxy | |
| | Molecule | chromanol | | chromanol | |

Table of commercially available Plant Extracts

| | | | % | | | |
|---|---|---|---|---|---|---|
| | | Active | active | Active | | Extraction |
| Supplier | Trade name | INCI | INCI | compound | Excipients | solvent |
| Indena/ | Centevita ™ | Centella | 100% | Asiatic and | None | None |
| Givaudan | | asiatica leaf | | madecassic acid | | |
| | | extract | | 45 to 100% | | |
| Croda | Phytessence ™ | Quercus | 5-10% | Polyphenols | Water 50-70%, | Ethanol/water |
| | French | Petraea fruit | | 0.70-1.10% | Glycerin 25- | |
| | Oak | extract | | | 50%, Sodium | |
| | | | | | benzoate approx | |
| | | | | | 0.6%, Citric | |
| | | | | | acid approx | |
| | | | | | 0.04% | |
| Merck | Emblica ® | Phyllanthus | 100% | Contains min. | 100% pure | |
| | | Emblica | | 60% low | | |
| | | fruit Extract | | molecular | | |
| | | | | weight | | |
| | | | | hydrolysable | | |
| | | | | tannins, | | |
| | | | | Emblicanin A & | | |
| | | | | Emblicanin B | | |
| | | | | being the key | | |
| | | | | bio-active | | |
| | | | | ingredients | | |
| Clariant | Red Snow ® | Camellia | 1-5% | Protocatechuic | Propanediol 30- | Propanediol/ |
| | | Japonica | | acid min. | 70%, Aqua 30- | water |
| | | flower | | 200 ppm | 70% | |
| | | extract | | | | |
| Naturex | Botany | Ginkgo | 100% | Ginkgoflavon- | 100% pure | Hydroalcoholic |
| | Ginkgo ™ | Biloba leaf | | glucosides 24- | | |
| | | extract | | 30%, Terpene | | |
| | | | | lactones 6-9% | | |
| Silab | Desoxine ® | Myrtus | 2.65% | polyphenols | water 97.05%, | |
| | Bio | Communis | | 2.5 g/1-5.0 g/l | Sodium | |
| | | leaf extract | | | benzoate 0.3% | |
| BASF | Arganyl ® LS | Argania | Approx. | Flavonoides | Glycerin | Glycerin/ |
| | 9781 | Spinosa leaf | 5.00% | 0.30-0.45% | 47.00%, | water |
| | | extract | | | Phenoxyethanol | |
| | | | | | 2.00%, Water to | |
| | | | | | 100% | |
| Horphag | Pycnogenol ® | Pinus | 100% | Procyanidins | 100% pure | |
| Research | | Pinaster | | 65-75% | | |
| | | back extract | | | | |
| Naturex | Green Tea | Camellia | 100% | Polyphenols >95%/ | 100% pure | n/a |
| | PE ™ | sinensis leaf | | Catechins >70% | | |
| | | extract | | | | |
| Silab | Cohesium ® | Ophiopogon | 9-13% | Sugars | Water | Water |
| | | japonicus | | (Fructose): | | |
| | | root extract | | 85-127 g/L | | |

1. Cosmetic composition suitable for use in application to menopausal skin comprising extract of Ophiopogon japonicus and at least one ceramide.

2. A composition according to claim 1 wherein said extract is achieved from the root of the Ophiopogon japonicus plant.

3. A composition according to claim 1 where the extract of Ophiopogon japonicus comprises a steroidal glycoside known as Ophiopogonin D (OP-D).

4. A composition according to claim 1 wherein the ceramide is selected from the group consisting of ceramide 1 (ceramide EOP), ceramide 2 (ceramide NG), ceramide 3 (ceramide NP), ceramide 6 (ceramide AP) and mixtures thereof.

5. A composition according to claim 1 wherein the ceramide is selected from the group consisting of ceramide 3 (ceramide NP), also known as N-oleoyl-4-hydroxysphinganine, ceramide 6 (ceramide AP) and mixtures thereof.

6. A composition according to claim 1 additionally comprising a phytoestrogenic isoflavone is derived from a phytoestrogenic plant selected from the group consisting of soyabean (Glycine max), red clover (Trifolium pratense), white clover (Trifolium repens), alfalfa (Medicago sativa), garlic (Allium sativum), celery (Apiaceae graveolens), carrot (Daucus carota), potato (Solanum tuberosum), rice (Oryza sativa), wheat (Triticum aestivum), sweet potato (Ipomoea batatas), apple (Malus domestica), pomegranate (Punica granatum), chaste berries (Vitex agnus-castus), coffee (Coffea arabica and Coffea robusta) and mixtures thereof.

7. A composition according to claim 1 additionally comprising a phytoestrogenic isoflavone selected from the group consisting of daidzein, genistein, glycitin, malonyldaidzein, malonylgenistein, malonylglycitin, acetyldaidzein and acetylglycitin, daidzein, genistein, glycitein and mixtures thereof.

8. A composition according to claim 1 additionally comprising a phytoestogenic isoflavone is selected from the group consisting of genistein (5, 7, 4′-Trihydroxy-Isoflavon), genistein (5, 7, 4′-Trihydroxy-isoflavone-7-glucoside), daidzein (7.4-Dihydroxy-Isoflavon), equol (4′, 7-Dihydroxyisoflavan), daidzein (7.4-Dihydroxy-isoflavone-7-glucoside), biochanin A (5.7-Dihydroxy-4′-of-methoxy-Isofla), glycitein (7.4-dihydroxy-6-methoxy-Isoflavon), glycitin (7.4-Dihydroxy-6-methoxy-isoflavone-7-glucoside), orobol (5.3, 4′-Trihydroxy-7-methoxy-Isoflavon), O-robol (5, 7, 3′, 4′-Tetrahydroxy-Isoflavon), santal (5, 7, 3′-Trihydroxy-4′-methoxy-Isoflavone) and/or enzyme inhibitors (5.4-Dihydroxy-7-methoxy-Isoflavone) and mixtures thereof.

9. A composition according to claim 1 additionally comprising a vitamin B or vitamin B derivative.

10. A composition according to claim 1 additionally comprising a vitamin B or vitamin B derivatives selected from nicotinic acid esters, including non-vasodilating esters of nicotinic acid, nicotinyl amino acids, nicotinyl alcohol esters of carboxylic acids, nicotinic acid N-oxide, niacinamide N-oxide and mixtures thereof.

11. A cosmetic composition according to claim 9 wherein the vitamin B or vitamin B derivative is niacinamide.

12. A method of cosmetically treating skin by application of a composition according to claim 1.
